(19) United States
(12) Patent Application Publication
(10) Pub. No.: US 2010/0028463 A1
(43) Pub. Date: Feb. 4, 2010

(54) EXTRACT OF STEWARTIA KOREANA AND USE THEREOF

(75) Inventors: Ji Young Kim, Seoul (KR); Dae Kyun Chung, Gyeonggi-do (KR); Tae Hoon Lee, Gyeonggi-do (KR); Guy Wilhem Lee, Gyeonggi-do (KR); Jung Min Lee, Gyeonggi-do (KR)

Correspondence Address: MOORE & VAN ALLEN PLLC, P.O. BOX 13706, Research Triangle Park, NC, 27709, US

(73) Assignee: RNA, INC., Gyeonggi-do (KR)

(21) Appl. No.: 12/279,194

(22) PCT Filed: Feb. 13, 2007
(86) PCT No.: PCT/KR2007/000763

§ 371 Date: Oct. 16, 2009

(30) Foreign Application Priority Data
Feb. 13, 2006 (KR) 10-2006-0013888

(51) Int. Cl. A61K 36/18; A61P 17/16

(52) U.S. Cl. 424/725

(57) ABSTRACT

The present invention relates to an extract of Stewartia koreana and use thereof. More particularly, it relates to an extract of Stewartia koreana which is extracted with any one selected from the group consisting of water, a C₁₋₄ low alcohol, a polar solvent, a non-polar solvent and a mixture thereof, and a pharmaceutical composition for promoting angiogenesis or tissue regeneration and a cosmetic for improving wrinkles comprising the same as an effective ingredient. The extract of Stewartia koreana according to the present invention promotes migration and multiplication of endothelial cell and shows excellent effect in angiogenesis and wound healing and thus, is useful in treatment or prevention of diseases which requires angiogenesis for healing of wounded and frostbitten region, wound healing after surgical operation, and treatment and prevention of gastric ulcer, ischaemic heart diseases and hair loss. Also, the extract shows the dermal tissue regenerating effect and thus, is useful as a cosmetic for improving wrinkles.

### TECHNICAL FIELD

The present invention relates to an extract of Stewartia koreana and use thereof. More particularly, it relates to an extract of Stewartia koreana which is extracted with any extraction solvent selected from the group consisting of water, a C₁₋₄ low alcohol, a polar solvent, a non-polar solvent and a mixture thereof, and a pharmaceutical composition for promoting angiogenesis or wound healing and tissue regeneration and a cosmetic for improving wrinkles comprising the same as an effective ingredient.

### BACKGROUND ART

The skin plays a role to protect the body from outer environment and maintain homeostasis of inner environment. When the skin is damaged, problems such as secondary inflammation with side effects may occur and thus, the treatment of the damaged skin is very important. If the wounded region is small and limited to the epidermis, the healing process includes steps of an initial inflammatory reaction, multiplication and migration of the epithelial cells, and reconstruction of the epidermis, whereby functions of normal cells are recovered. If the basement membrane of the skin is damaged, the damaged part is recovered by undergoing the inflammation step, in which a thrombus is formed, extracellular substrates such as fibrin, fibronectin and hyarylonic acid are deposited and impurities and necrotic tissue in the wound are removed to clean the wound, the multiplication step, in which blood vessels are formed, growth factors such as PDGF, EGF, FGF and the like are released, the wound region was filled with new tissue and supplemented with new epithelial cells, and the maturation step, in which the collagen bundle is deformed and reconstructed, the wound region shrinks and the tension is increased (Stadelmann, W. K. et al., Am J. Surg., 26S(176), 1998).

The wound healing begins with the process of filling the damaged tissue with 5 matrix collagen, fibronectin and the like, in which collagen synthesis plays an important role. By playing an important role in the wound healing or skin regeneration, the collagen promotes wound healing and skin regeneration, and inhibits scar formation (Ueno, H. et al., Biomaterial, 1407(20), 1999; Buckley, A. et al., Proc. Natl. Acad. Sci., 7340(82), 2000; Manxi, L. et al., Cell Tissue Res., 423(297), 1999).

In the wound healing process as described above, the most important phenomenon is angiogenesis. The angiogenesis represents a series of procedures, in which new blood vessels are formed from existing blood vessels. By the angiogenesis, when a new tissue is formed in the course of the wound healing, oxygen and nutrients are supplied to the new tissues so that the tissue can be regenerated as a part of the body to carry out their functions.

When the skin gets wounded, fibroblast growth factors are excessively expressed from fibroblasts of the damaged tissue. By this, endothelial cells are activated and proteolytic enzyme of extracellular substrate protein is expressed from endothelial cells. As a result, the endothelial cells degrade the extracellular substrates to provide path where new blood vessels are formed and undergo permeation and migration. The migrated endothelial cells are differentiated to maintain their own functions and the endothelial cells after the differentiation form a vessel to supply growth factors, oxygen and nutrients, which is not a tube with a perfect shape like the original vessel. That is, the cells damaged by wound or necrosis are supplied with new nutrients through new blood vessels, and fibrocyte and epidermal cells differentiate and grow with the supplied oxygen and nutrient, whereby tissue reproduction of the wound tissue and skin regeneration is completed.

Meanwhile, Stewartia koreana is a deciduous broad-leaves tree belonging to dicotyledonous plants of Order Guttiferales of family Theaceae and is distributed in Korea and Japan growing over the middle slope of a mountain. Stewartia koreana has a height of about 7 to 15 m and its bark is dark reddish brown and stripped off in large pieces, which becomes smooth like Lagerstroemia indica when it is old. Its leaves are alternate phyllotaxis and oval or broad oval with a sharp end and a round or blunt base. The leaves have a length of4 to 10 cm and a width of2 to 5 cm with saw tooth edge in a wave shape.

Stewartia koreana has a white bisexual flower hung at the lower part of a new stem and blooming June to July. The flower stalk has a length of 1.5 to 2 cm and the bract is egg shaped or round. The calyx is round and has villi and the petal is upside down egg shaped and 5 to 6. The pistil is divided into 5 and combined and the stamen is 5. The fruit of Stewartia koreana is a capsule type pentagonal pyramid and is ripe in October. The timber is used for ornament and high quality furniture.

It has been known for a long time that intake of Stewartia koreana stem extract is extremely effective in treatment of various liver diseases such as liver inflammation, liver cirrhosis, fatty liver and paralysis of all 4 limbs. However, scientific and systematic research on the extract of Stewartia koreana and its leaves is not yet insufficient.

Thus, the pertinent field has required continuous study and development of the extract of Stewartia koreana and its leaves effective in various diseases.

Accordingly, the present inventors have conducted researches on the effects of the Stewartia koreana extract and found that the extract of Stewartia koreana leaves promotes migration and multiplication of endothelial cells and shows excellent effect in angiogenesis, wound healing and regeneration of dermal tissues. Based on the above founding, the present invention has been completed.

### SUMMARY OF THE INVENTION

The main object of the present invention is to provide an extract of Stewartia koreana showing the angiogenesis promoting effect.

Another object of the present invention is to provide a pharmaceutical composition for treatment or prevention of diseases which requires angiogenesis for healing of wounded and frostbitten region and wound healing after surgical operation, and treatment and prevention of gastric ulcer, ischaemic heart diseases and hair loss, which comprises the extract of Stewartia koreana.

A further object of the present invention is to provide a cosmetic for improving wrinkles, which comprises the extract of Stewartia koreana.

In order to accomplish the above objects, the present invention provides an extract of Stewartia koreana showing the angiogenesis promoting effect.

In the present invention, said extract of Stewartia koreana is preferably extracted with any one selected from the group consisting of water, a C₁₋₄ low alcohol, a polar solvent, a non-polar solvent and a mixture thereof. In particular, the extract of Stewartia koreana is preferably prepared by the following steps: (a) adding leaves of Stewartia koreana in any extraction solvent selected from the group consisting of water, a C₁₋₄ low alcohol, a polar solvent, a non-polar solvent and a mixture thereof and performing extraction under reflux at 40 to 80° C. while stirring; (b) isolating a filtrate by filteration of the extract; and (c) obtaining a powder by concentrating the filtrate at reduced pressure.

Also, the mixture of solvents is preferably a 30 to 99% ethanol aqueous solution or a 30 to 99% methanol aqueous solution.

The present invention also provides a pharmaceutical composition for promoting angiogenesis and a pharmaceutical composition for promoting tissue regeneration in the wounded region, comprising the extract of Stewartia koreana as an effective ingredient.

The present invention also provides a cosmetic for improving wrinkles, comprising the extract of Stewartia koreana as an effective ingredient.

The above and other objects, features and embodiments of the present invention will be more clearly understood from the following detailed description and accompanying claims.

### BRIEF DESCRIPTION OF THE DRAWINGS

FIG. 1 is a graph showing the migration of endothelial cells by the extract of Stewartia koreana leaves according to the present invention and a conventional therapeutic agent for wound healing.

FIG. 2 is a graph showing the effect of the extract of Stewartia koreana leaves according to the present invention on multiplication of endothelial cells.

FIG. 3 shows the angiogenesis promoting effect of the extract of Stewartia koreana leaves according to the present invention in egg (CAM).

FIG. 4 is a view showing the number of blood vessels in egg, formed by the extract of Stewartia koreana leaves according to the present invention.

FIG. 5 is a view showing the wound healing effect in mouse by the extract of Stewartia koreana leaves according to the present invention.

FIG. 6 is a graph showing the change in wound size of mouse by the extract of Stewartia koreana leaves according to the present invention.

FIG. 7 is a view showing the wounded tissue regeneration effect by the extract of Stewartia koreana leaves according to the present invention.

FIG. 8 is a graph showing the MMP-1 activity inhibition by the methanol extract of Stewartia koreana according to the present invention at various concentrations.

FIG. 9 is a graph showing the MMP-1 activity inhibition by the ethyl acetate fraction of the extract of Stewartia koreana according to the present invention.

### DETAILED DESCRIPTION OF THE INVENTION, AND PREFERRED EMBODIMENTS

In one aspect, the present invention relates to an extract of Stewartia koreana showing the angiogenesis promoting effect.

The extract of Stewartia koreana according to the present invention is prepared by extracting from leaves of Stewartia koreana with an extraction solvent selected from water, a C₁₋₄ low alcohol, a polar solvent, a non-polar solvent and a mixture thereof. Concretely, the leaves of Stewartia koreana are dried, pulverized and extracted with water, a low alcohol such as methanol, ethanol and butanol or a 1:0.1 (v:v) to 1:10(v:v) mixture thereof, preferably water or about 70% (v) ethanol or methanol, in an amount of 5 to 25 times, preferably about 10 times, of the dry weight at an extraction temperature of 20 to 100° C., preferably 40 to 80° C., for about 30 minutes to 2 days, preferably 1 hour to 1 day, by hot water extraction, cold dipping extraction, reflux cooling extraction or ultrasonic extraction, in a continuous way of 1 to 5 times, preferably 2 to 3 times, followed by filtration with filter paper. The resulting filtrate is vacuum-concentrated using a rotary evaporator at 20 to 80° C., preferably 40 to 60° C., and dried by vacuum freeze drying, hot-air drying or spray-drying to obtain powder of the Stewartia koreana extract.

As a solvent for extraction, water, low alcohol such as, methanol, ethanol and buthanol, or a mixture thereof is preferably used. However, a polar solvent such as 1-pentanol, 2-butoxyethanol, 1-propanol, 2-propanol, ethylene glycol, acetic acid, DMFO, DMSO and the like; and a non-polar solvent such as acetone, acetonitrile, ethyl actate, methyl acetate, fluoroalkanes, pentanes, hexane, 2,2,4-trimethylpentane, decane, cyclohexane, cyclopentane, diisobutylene, 1-pentene, 1-chlorobutane, 1-chloropentane, o-xylene, diisopropyl ether, 2-chloropropane, toluene, 1-chloropropane, chlorobenzene, benzene, diethyl ether, diethyl sulfide, chloroform, dichloromethane, 1,2-dichloroethane, dimethyl sulfoxide, aniline, diethylamine, ether, CCl₄, THF, and the like can be also used.

In order to examine the wound healing effect of Stewartia koreana extract, the effect of Stewartia koreana extract on migration and differentiation of endothelial cells, the effect on angiogenesis and the effect on the wound healing were examined and it has been found that those effects were excellent. Particularly, it has been noted that the inventive extract of Stewartia koreana can completely recover damaged tissue without any scar after wound healing.

Therefore, the present invention, in another aspect, relates to a pharmaceutical composition for promoting angiogenesis and a pharmaceutical composition for promoting tissue regeneration in the wounded region, comprising the extract of Stewartia koreana as an effective ingredient.

The pharmaceutical composition for recovering a wound according to the present invention can be formulated into a common pharmaceutical formulation according to a method known to the pharmaceutical field. Preferred examples of the pharmaceutical formulation include formulations for oral administration such as tablet, hard or soft capsules, liquid, suspension and the like, formulations for injection, and formulations for topical administration such as ointment, cream, gel, lotion and the like. Such pharmaceutical formulations can be prepared using a pharmaceutically acceptable carrier, for example an excipient, a binder, a disintegrant, a lubricant, a solubilizing agent, an emulsifying agent, a preservative or an extending agent in case of the formulations for oral administration, a stabilizer, a preservative, a dissolving adjuvant, a buffer, an isotonizing agent in case of the formulations for injection, and aqueous or oily ointment base, an antioxidant, a preservative, an extending agent and the like in the case of the formulation for external use.

The preferred dose of Stewartia koreana extract according to the present invention may be properly selected by the person in the art according to the condition and weight of the patient, disease severeness, drug type, and route and period of administration. However, for optimum result, the extract is administered in an amount of 0.01 to 1 g/kg, preferably 0.05 to 0.5 g/kg, per day. The administration is performed once to several times per day. The foregoing dose does not limit the scope of the invention in any way.

The extract of Stewartia koreana according to the present invention may be used in a cosmetic for improving wrinkles since it shows dermal tissue regeneration effect. Therefore, the present invention, in still another aspect, relates to a cosmetic for improving wrinkles, comprising the extract of Stewartia koreana as an effective ingredient.

One of the reasons of dermal wrinkle formation is lack of extracellular matrix such as collagen and elastin. Collagen is a main protein forming the dermis, playing a role to maintain the structure and elasticity of the skin. The production of collagen and elastin is reduced and degradation thereof is increased by environmental factors and aging, and thus, the polymer network structure of the skin is damaged. Ultimately, the elasticity and restitution force of the skin is reduced, thereby causing wrinkle formation. Therefore, the conventional cosmetic products for improving wrinkles are mainly those comprising retinol promoting multiplication of epithelial cell and endothelial cell and synthesis of collagen which is a component of the dermis. This is because collagen plays an important role in regeneration of dermal tissues including promotion of multiplication of fibroblasts, promotion of angiogenesis of damaged cite of the skin and induction of secretion of other regeneration-promoting factors, and promotion of synthesis of fibronectin which is a substance allowing dermal tissues to form a net with orderly orientation.

The inventive extract of Stewartia koreana also shows angiogenesis effect similar to that of collagen and is closely related to regeneration of dermal tissue, and thus is useful as a cosmetic product for improving wrinkles.

According to the present invention, the cosmetic product for improving wrinkles includes any formulation selected from the group consisting of skin softener, cream, lotion, skin lotion, pack, foundation, liquid soap, solid soap and cleansing foam.

### Examples

The present invention will hereinafter be described in further detail by examples. However, it is to be understood that these examples can be modified into other various forms, and the scope of the present invention is not intended to be limited to such examples. Such examples are given to more fully describe the present invention for a person skilled in the art.

### Example 1

### Preparation of Stewartia koreana Extract

### 1-1: Hot Water Extraction using Water

Stewartia koreana leaves were collected, washed, dried and pulverized. 1 kg of the pulverized leaves was added to 10 L of water and extracted under reflux at 60 to 65° C. for 5 hours while stirring and filtered to obtain the filtrate. The filtrate was vacuum concentrated at 55 to 65° C., lyophilized to give 165 g of the Stewartia koreana extract as dry powder.

### 1-2: Hot Water Extraction using Water-Ethanol Mixture

1. of the Stewartia koreana leaves pulverized as in Example 1-1 was added to 10 L of 30% (v/v), 50% (v/v) and 70% (v/v) ethanol aqueous solution, extracted under reflux at 60 to 65° C. for 5 hours while stirring and filtered to obtain the filtrate. The filtrate was vacuum concentrated at 55 to 65° C., lyophilized to give 135, 154 and 178 g of the Stewartia koreana extract as dry powder.

### 1-3: Extraction using Water-Methanol Mixture

1. of the Stewartia koreana leaves pulverized as in Example 1-1 was added to 10 L of 80% (v/v) methanol aqueous solution, dip-extracted at room temperature for 24 hours while stirring and filtered to obtain the filtrate. After this procedure was repeated two times, the resulting filtrate was vacuum concentrated at 55 to 65° C., lyophilized to give 1178 g of the Stewartia koreana extract as dry powder.

### Example 2

### Preparation of Stewartia koreana Extract using Polar and Non-Polar Solvent

### 2-1: Separation of Chloroform Soluble Fraction

50 g of the extract obtained by the hot water extraction in Example 1-1 was dissolved in 500 mL of water and poured into a separatory funnel, in which it was vigorously mixed with 500 mL of chloroform to separate the aqueous layer and the chloroform soluble layer. The aqueous layer and the chloroform layer were vacuum concentrated at 70 to 75° C. and 45 to 50° C., respectively, and lyophilized.

### 2-2: Separation of ethyl acetate Soluble Fraction

To the chloroform soluble layer obtained in Example 2-1, ethyl acetate was added in the same volume and poured into a separatory funnel, in which the mixture was vigorously mixed to separate the ethyl acetate soluble fraction and insoluble fraction. The ethyl acetate soluble fraction was vacuum concentrated and lyophilized.

### 2-3: Separation of ethyl acetate Soluble Fraction of Stewartia koreana Methanol Extraction

1000 g of methanol extract obtained by dip extraction in Example 1-3 was put into a separatory funnel with 3000 mL of water and 3000 mL of ethyl acetate and vigorously mixed together to separate the aqueous layer and ethyl acetate soluble layer. The aqueous layer and the ethyl acetate layer were vacuum concentrated at 70 to 75° C. and 45 to 50° C., respectively, and lyophilized.

### 2-4: Separation of Sub-Fraction of ethyl acetate Soluble Fraction

300 g of ethyl acetate soluble fraction obtained in Example 2-3 was eluted by silica gel column chromatography (φ12×20 cm) with chloroform:methanol of 12:1, 10:1, 7:1 and 5:1 as an eluting solvent and each fraction was separated by TLC (Thin-Layer Chromatography).

Upon carrying out TLC, fraction 1 (Rf: 1 to 0.9), fraction 2 (Rf: 0.9 to 0.7), fraction 3 (Rf: 0.7 to 0.5) and fraction 4 (Rf: 0.5 to 0.3) were separated with the chloroform/methanol ratio of 8:1 and fraction 5 (Rf: 0.7 to 0.5), fraction 6 (Rf: 0.5 to 0.4) and fraction 7 (Rf: 0.4 or less) were separated with the chloroform/methanol ratio of 4:1.

### Example 3

### Effect of Stewartia koreana Extract on Migration of Endothelial Cells

The migration of endothelial cells is an indispensable step in the angiogenesis and the effect of the Stewartia koreana extract according to the present invention on the migration of endothelial cells was confirmed by the Boyden chamber method (Gho, Y. S. et al., Cancer Res., 59:5128, 1999). 0.1% gelatin was applied on a polycarbonate membrane (Costar, USA) for 10 minutes and dried at room temperature for 1 hour. 30 μL of human umbilical vein endothelial cells (HUVEC, 1×10⁶ cells/mL) was put in a lower chamber of a trans-wells (Costar, USA), covered with the dried polycarbonate membrane, and an upper chamber was overlaid, followed by fixing by a screw. The chamber was turned over so that the cells could be attached to the membrane and cultured at 37° C. in a CO₂ incubator for 2 hours.

### 3-1: Counting the Number of the Migrated Endothelial Cells

50 μL of Stewartia koreana extract according to the present invention was added to the upper chamber and cultured for 2 hours. The polycarbonate membrane was died using the diff-quick staining agent and the migrated endothelial cells were counted through the polycarbonate membrane using a microscope (Table 1). Each dry extract powder of Stewartia koreana otained in Example were dissolved in water in an amount of 100 μg/mL. The negative control was water and the positive control was 10 ng/mL of VEGF (vascular endothelialgrowth factor, Sigma). As a result, as shown in Table 1, the extract of Stewartia koreana leaves according to the present invention effectively induced migration of endothelial cells and particularly, the extract with 70% ethanol and 70% ethanol by hot water extraction was the most effective.

TABLE 1

| | Migrated |
|---|---|
| Treatment | endothelial cells |
| Water (negative control) | 20 ± 0.3 |
| VEGF (positive control) | 75 ± 0.41 |
| Extract by hot water extraction | 45 ± 0.22 |
| 10% ethanol extract by hot water extraction | 27 ± 0.32 |
| 30% ethanol extract by hot water extraction | 39 ± 0.36 |
| 50% ethanol extract by hot water extraction | 51 ± 0.35 |
| 70% ethanol extract by hot water extraction | 79 ± 0.29 |
| 100% ethanol extract by hot water extraction | 65 ± 0.35 |
| 10% methanol extract by hot water extraction | 25 ± 0.36 |
| 30% methanol extract by hot water extraction | 43 ± 0.39 |
| 50% methanol extract by hot water extraction | 54 ± 0.25 |
| 70% methanol extract by hot water extraction | 83 ± 0.31 |
| 100% methanol extract by hot water extraction | 67 ± 0.25 |
| Chloroform soluble fraction | 40 ± 0.37 |
| Ethyl acetate soluble fraction | 48 ± 0.48 |
| Water fraction | 19 ± 0.14 |

### 3-2: Counting of the Number of Migrated Endothelial Cells

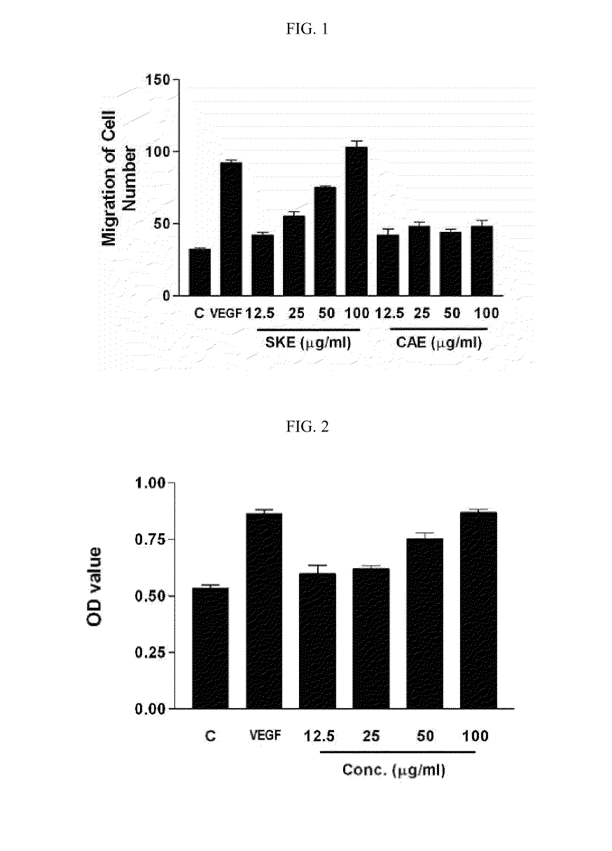

50 μL of 70% ethanol extract of Stewartia koreana by hot water extraction and Compound Madecassol (Centella asiatica extract, Dongkook Pharm Co., Ltd., Korea) were added to the upper chamber prepared as the above and cultured for 2 hours. The polycarbonate membrane was stained with diff-quick staining agent and the number of the endothelial cells which had migrated through the polycarbonate membrane was then counted using a microscope (FIG. 1). In FIG. 1, C and VEGF represent negative control (water) and positive control (VEGF 10 ng/mL), respectively, SKE and CAE represent dry powder of the ethanol extract of Stewartia koreana and compound Madecassol™. The dry powder of the ethanol extract of Stewartia koreana and compound Madecassol™ were added to water in an amount of 12.5 to 100 μg/mL. As a result, it was confirmed that the ethanol extract of Stewartia koreana leaves according to the present invention more effectively induced the migration of endothelial cells, as compared to compound Madecassol™, as shown in FIG. 1. Particularly, it was shown that the 70% ethanol extract of Stewartia koreana by hot water extraction, when added in a concentration of 100 μg/mL, more effectively promoted the migration of endothelial cells than VEGF, which is known as a strong angiogenesis inducing factor.

### Example 4

### Effect of Stewartia koreana Extract on Multiplication of Endothelial Cells

The effect of Stewartia koreana extract on the multiplication of endothelial cells in the course of angiogenesis was examined by the Boyden chamber method. 0.3 mL of the 1:1 (v:v) mixture of matrigel (BD Bioscience) and serum-free RPMI 1640 medium (Hyclone) was added in a 24-well plate and solidified in an incubator at 37° C. for 1 hour. Then, umbilical vein endothelial cells (4×10⁴/well) and dry powder of the 70% ethanol extract by hot water extraction were added to each well at different concentrations and cultured in a CO₂ incubator at 37° C. for 48 hours. The number of the multiplied cells was measured (FIG. 2). In FIG. 2, C and VEGF represent negative control (water) and positive control (VEGF 10 ng/mL), respectively.

As a result, it was shown that the 70% ethanol extract of Stewartia koreana by hot water effectively induced the multiplication of endothelial cells, as shown in FIG. 2, and, when added in a concentration of 100 μg/mL, showed similar effect to VEGF, which is known as a strong angiogenesis inducing factor.

### Example 5

### Angiogenesis Promoting Effect of Stewartia koreana Extract In vivo

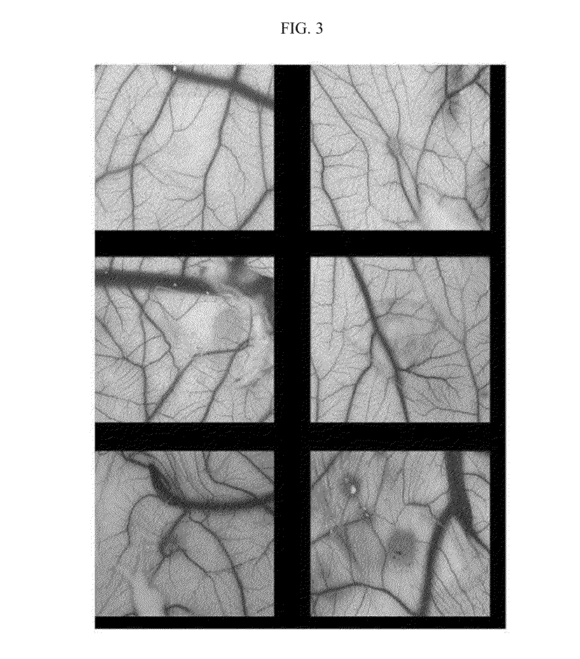

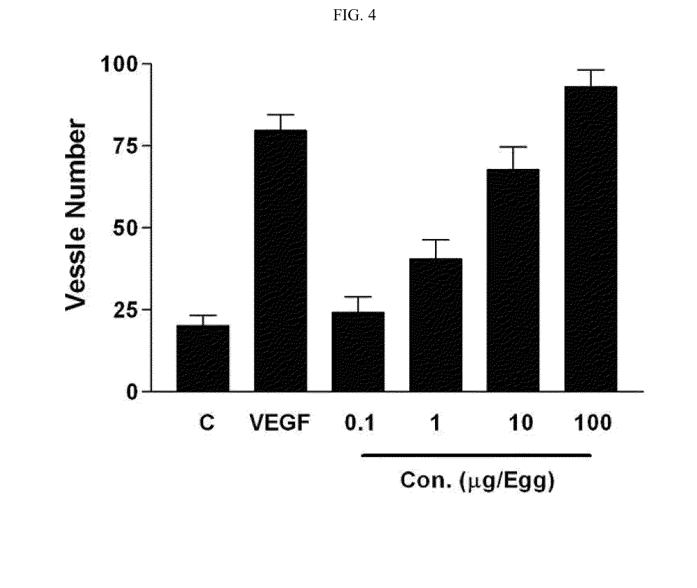

The angiogenesis promoting effect of the 70% ethanol extract of Stewartia koreana by hot water extraction was examined by the CAM (chorioallantoic membrane) test method (Gho, Y. S. et al, Cancer Res., 59:5128, 1999). A fertilized egg was cultured at 37° C. for 2 days. 4 mL of albumin was removed and after four days, the egg shell of 3 cm×3 cm was removed to make a hole. The eggs were further cultured. 18 μL of the mixture of type I collagen (Rat tail, Becton Dickinson, USA) and the 70% ethanol extract of Stewartia koreana by hot water extraction was applied dropwise on Thermanox coverslip and dried. At 10^{th} day, the thermanox coverslip was laid upon the CAM of the egg, cultured for 3 days. The number of blood vessels newly formed by the extract of Stewartia koreana was counted (FIG. 3 and FIG. 4). In FIG. 4, C and VEGF represent negative control (water) and positive control (VEGF 10 ng/mL), respectively.

As a result, as shown in FIG. 3 and FIG. 4, upon treatment of the 70% ethanol extract by hot water extraction at a concentration of 100 μg/egg, the number of the newly formed blood vessels was larger than that of VEGF, which is known as a strong angiogenesis inducing factor.

### Example 6

### Wound Healing Effect in Mouse

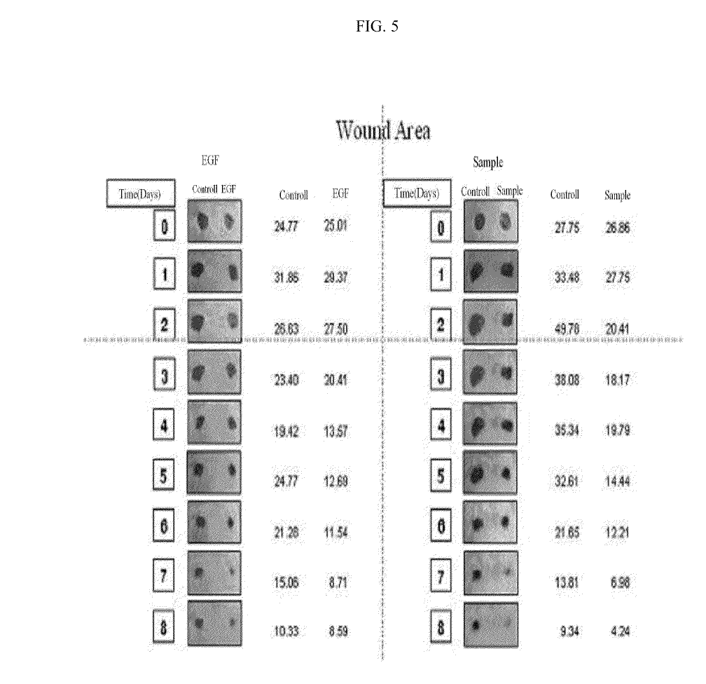

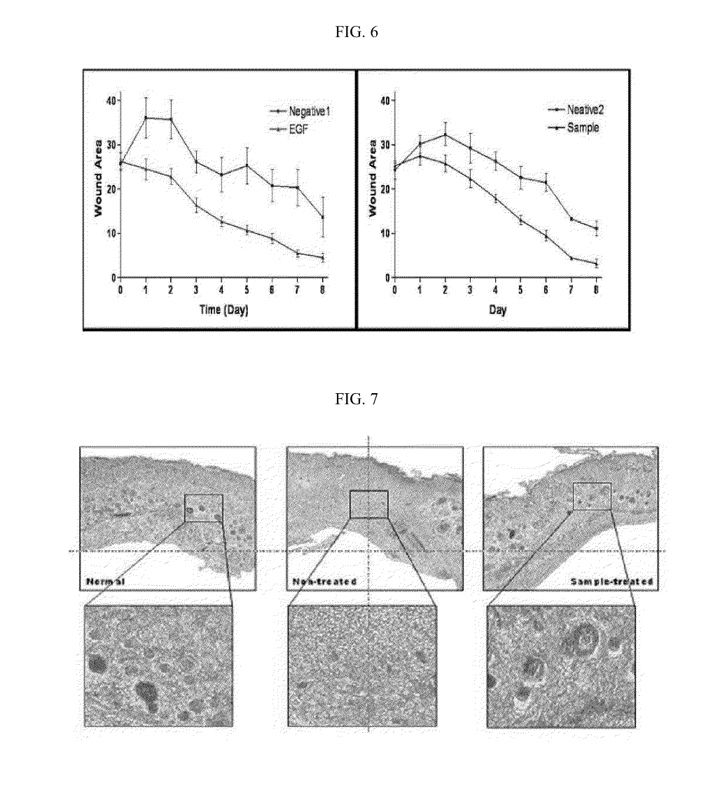

In order to examine the wound healing effect of the 70% ethanol extract of Stewartia koreana by hot water extraction, a part of the skin of a mouse was inflicted with a wound of 6 mm and the wound healing was observed. The experiment was performed according to prior reference (Repertinger, S. K. et al., J. Invest. Dermatol., 982(123), 2004) and the animals were observed for 9 days. Each mouse was wounded at two sites and the mice were compared to each other. The negative control was PBS and the positive control was EGF (50 ng/mice). Each mouse was treated with 200 μg of the extract of Stewartia koreana. The sample was treated at the same time everyday for 9 days. The healed area was calculated by scion image analysis (FIG. 5 and FIG. 6).

As a result, as shown in FIGS. 5 and 6, the wound area was rapidly recovered after 3 days in the treatment group. It was confirmed that the final wound area of the sample treated with the 70% ethanol extract by hot water extraction according to the present invention was smaller than that of the group treated with EGF.

### Example 7

### Tissue Regeneration Effect in Wounded Tissue

In order to examine the tissue regeneration effect in a wounded tissue of the 70% ethanol extract of Stewartia koreana by hot water extraction, tissue staining test was performed. The experiment was performed using the H.E. staining method according to a reference (Repertinger, S. K. et al., J. Invest. Dermatol., 982(123), 2004). The tissue obtained by incising the wound margin of a mouse which had been recovered at least 80% or more was fixed in the 4% PFA solution for 12 hours, dehydrated and solidified at room temperature for 24 hours. The resulting sample was cut into a 10 μm segment, placed on a slide and stained by using the H.E solution, followed by observation. Here, the extracellular tissues were stained red and the cell was stained blue. As a result, as shown in FIG. 7, it was confirmed that when samples were treated with the 70% ethanol extract by hot water extraction, the angiogenesis occurred actively.

### Formulation Examples

Examples of pharmaceutical formulations using the extract of Stewartia koreana which is excellent in angiogenesis and wound healing as described in Examples were described. However, it is only for the illustrative purpose and the present invention is not limited thereto.

### Formulation 1: Preparation of Injection Formulation

100 mg of the 70% ethanol extract by hot water extraction from Example 1-2; 3.0 mg of sodium methabisulfite; 0.8 mg of methyl paraben; and 0.1 mg of propyl paraben were mixed with sterilized distilled water for injection to make the total volume of 2 mL, filled into an ample and sterilized.

### Formulation 2: Preparation of Tablet

300 mg of the 70% ethanol extract by hot water extraction from Example 1-2; 100 mg of lactose; 100 mg of starch; and a suitable amount of magnesium stearate were mixed together and tableted according to a known method.

### Formulation 3: Preparation of Capsule

300 mg of the 70% ethanol extract by hot water extraction from Example 1-2; 50 mg of lactose; 50 mg of starch; 2 mg of talc; and a suitable amount of magnesium stearate were mixed together and filled into gelatin capsule according to a known preparation method of capsule.

### Formulation 4: Preparation of Liquid Formulation

500 mg of the 70% ethanol extract by hot water extraction from Example 1-2; 5 g of sugar; 10 g of isomerized sugar; and a suitable amount of lemon flavor were mixed with purified water to be 100 mL, filled into a 100 mL of amber bottle and sterilized.

### Formulation 5: Preparation of Ointment

100 mg of the 70% ethanol extract by hot water extraction from Example 1-2; 100 mg of light liquid paraffin; 80 mg of stearyl alcohol; 13 mg of cetostearyl alcohol; 50 mg of propylene glycol; 30 mg sorbitan monostearate; 40 mg of polyoxyethylsorbitan monostearate; 0.4 mg of butylated hydroxytoluene; 0.9 mg of methyl paraoxybenzoate; 0.9 mg of butyl paraoxybenzoate; and a suitable amount of purified water were mixed together according to a known preparation method of ointment to prepare an ointment formulation containing 100 mg of the 70% ethanol extract by hot water extraction from Example 1-2 per 1 g.

Now, examples of the cosmetic formulations having the skin regeneration effect using the extract of Stewartia koreana are explained. However, it is only for the illustrative purpose and the present invention is not limited thereto.

### Formulation 6: Skin Softener

100 mg of the 70% ethanol extract of Stewartia koreana leaves by hot water extraction from Example 1-2; 250 mg of glycerin; 150 mg of 1,3-butylene glycol; 50 mg of PEG 1500; 5 mg of allantoin; 1.5 mg of DL-panthenol; 1 mg of EDTA; 2 mg of benzophenone-9; 250 mg of sodium hyaluronate; 500 mg of ethanol; 10 mg of Octyldodeceth-16; 10 mg of polysorbate; a preservative, flavor, 2 mg of a colorant; and a suitable amount of distilled water were mixed according to a known method to prepare a skin softener.

### Formulation 7: Preparation of Cream

75 mg of the 70% ethanol extract of Stewartia koreana leaves by hot water extraction from Example 1-2; 100 mg of self-emulsifying glycerin monostearate; 110 mg of stearyl alcohol; 75 mg of stearic acid; 50 mg of wax; 75 mg of polysorbate-60; 30 mg of sorbitan stearate; 50 mg of hardened vegetable oil; 150 mg of squalane; 250 mg of mineral oil; 250 mg of trioctanoyl; 50 mg of dimethicone; 5 mg of sodium magnesium silicate; 250 mg of glycerin; 150 mg of betain; 50 mg of triethanol amine; 200 mg of sodium hyaluronate; a preservative, flavor, 2 mg of a colorant; and a suitable amount of distilled water were mixed according to a known method to prepare a cream.

The above-described mixing ratios are made for preferred examples which are relatively suitable for favorite liking cosmetic products but may vary according to regional and ethnic preference such as the receving classes, receving nations and use thereof.

### Experimental Examples

In order to examine if the angiogenesis effect of the extract of Stewartia koreana leaves according to the present invention is applied to cosmetic products for 15 improving wrinkles, the wrinkle improving effect and stability tests were performed using the skin softener of Formulation 6.

### Experimental Example 1: Test of Wrinkle Improving Effect

### 1-1: Measurement of Change in Dermal Wrinkles

A piece of gauze was wet with the skin softener of Formulation 6 and applied on left and right upper arm of 10 subjects (30 or more years old women) in an area of 2×2 cm² for 6 weeks (twice per day). The change in dermal wrinkles was measured by making a replica using a transparent silicone based solution and observing with SKIN VISOMETER SV400 (C+K Electronics, Germany). The image of the replica was analyzed three-dimensionally using a CCD camera and the dermal wrinkle improving effect was analyzed using the value obtained by dividing the sum of wrinkle roughness (Rₘ: m is an integer of 1 or more) by the number of wrinkles, that is, the average wrinkle roughness (Rz), as shown in Equation I (Table 2).

Rz=(R₁+R₂+ . . . +Rₘ₋₁+Rₘ)/the number of wrinkles (m) Equation I:

TABLE 2

ΔRz = Rz before test − Rz after 6 weeks

| | Control | Skin softener of Formulation 6 |
|---|---|---|
| Subject 1 | 0.010 | 0.080 |
| Subject 2 | 0.021 | 0.085 |
| Subject 3 | 0.018 | 0.075 |
| Subject 4 | 0.010 | 0.083 |
| Subject 5 | 0.020 | 0.070 |
| Subject 6 | 0.013 | 0.079 |
| Subject 7 | 0.020 | 0.069 |
| Subject 8 | 0.012 | 0.078 |
| Subject 9 | 0.030 | 0.083 |
| Subject 10 | 0.020 | 0.090 |

In Table 2, the control was treated with gauze wet by a solution containing the components of Formulation 6 except for the extract of Stewartia koreana leaves. As a result, as shown in Table 2, the skin softener of Formulation 6 comprising the extract of Stewartia koreana leaves had far better wrinkle improving effect.

### 1-2: Inhibition Effect of the Methanol Extract (SKM) of the Stewartia koreana on MMP-1 Activity at Various Concentrations

Fibroblasts of a normal human were inoculated in a 24-well plate at 3×10⁵ cells/well, cultured until subconfluent, washed with PBS, treated with 5, 10 and 50 μg/μL of SKM obtained in Examples 1-3, and incubated for 24 hours. The cultured cells were washed with PBS buffer and total RNA was isolated using 1 mL of RNABee (TEL-TEST Inc) per well. cDNA was synthesized from the isolated total RNA (1 μg) using ImProm-II Reverse Transcription System (Promega).

1 μL of the synthesized cDNA, 1 μL of human MMP-1 derived primers of SEQ ID NOs: 1 and 2, and 10 μL of SYBR GREEN PCR Master Mix (Applied Biosystems) were mixed with distilled water to make the total volume of 20 μL and amplified by PCR. The PCR was performed under the following comditions: 2 minutes at 50° C., minutes at 95° C., and 45 cycles of 15 seconds at 95° C., 1 minute at 60° C. The amplified product was measured for fluorescence using ABI PRISM 7000 detection system (Applied Biosystems) (FIG. 8). As a result, as shown in FIG. 8, when the methanol extract (SKM) of the Stewartia koreana were added, the activity of MMP-1, a collagenase was rapidly reduced.

SEQ ID NO: 1: 5′-TGC GCA CAA ATC CCT TCT AC
SEQ ID NO: 2: 3′-TGT CCC TGA ACA GCC CAG TA

### 1-3: Inhibition Effect of the ethyl acetate Fraction (SKEA) of the Stewartia koreana Extract on MMP-1 Activity at Various Concentrations

Fibroblasts of a normal human were inoculated in a 24-well plate at 3×10⁵ cells/well, cultured until subconfluent, washed with PBS, treated with 25 μg/μL of SKEA obtained in Examples 2-3, 2.5 μMM of Retinol and 5 μg/μL of fractions 1˜7 separated in Examples 2-4 and incubated for 24 hours. The cultured cells were washed with PBS buffer and total RNA was isolated using 1 mL of RNABee (TEL-TEST Inc) per well. cDNA was synthesized from the isolated total RNA (1 μg) using ImProm-II Reverse Transcription System (Promega).

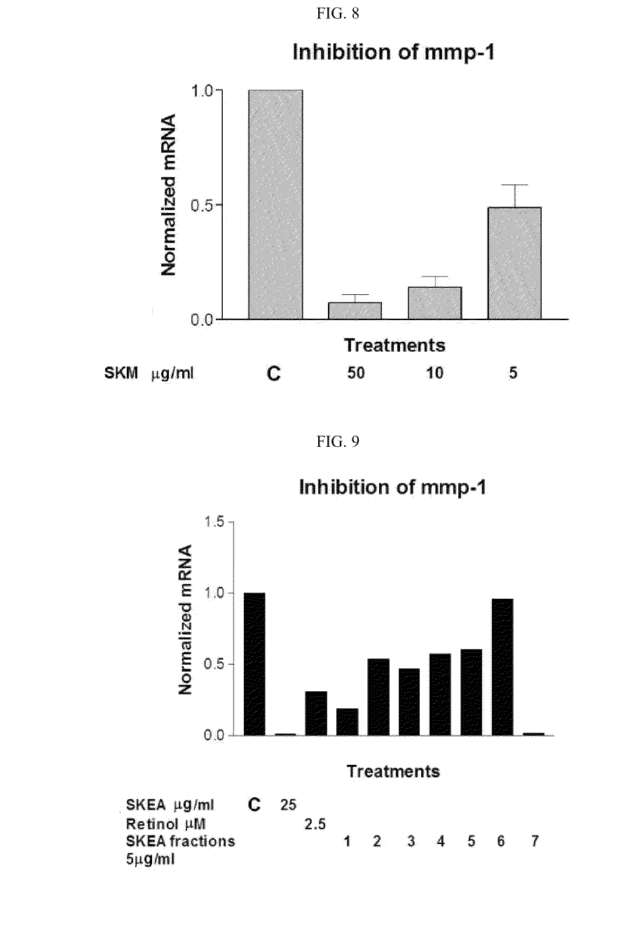

1 μL of the synthesized cDNA, 1 μL of human MMP-1 derived primers of SEQ ID NOs: 1 and 2, and 10 μL of SYBR GREEN PCR Master Mix (Applied Biosystems) were mixed with distilled water to make the total volume of 20 μL and amplified by PCR. The PCR was performed under the following comditions: 2 minutes at 50° C., 10 minutes at 95° C., and 45 cycles of 15 seconds at 95° C., 1 minute at 60° C. The amplified product was measured for fluorescence using ABI PRISM 7000 detection system (Applied Biosystems) (FIG. 9). As a result, as shown in FIG. 9, when SKEA and fractions 1˜7 isolated in Examples 2-4 were added, the activity of MMP-1, a collagenase was rapidly reduced. Particularly, it was noted that the fractions 1˜7 more reduced the activity of MMP-1 more than the group treated with retinol.

### Experimental Example 2

### Safety Test

The Human body safety test was performed using the skin softener of Formulation 6. 10 males and 20 females were subjected to Use test and Patch test using the skin softener of Formulation 6. As a result, the average irritation levels were 0.1 and 0.15, respectively and thus, all the subjects did not show irritation.

### INDUSTRIAL APPLICABILITY

As described above, the extract of Stewartia koreana according to the present invention promotes angiogenesis in vivo by inducing migration and differentiation of endothelial cells. Therefore, it shows excellent effect in treatment or prevention of diseases which requires angiogenesis for healing of wounded and frostbitten region and wound healing after surgical operation, and treatment and prevention of gastric ulcer, ischaemic heart diseases and hair loss. Also, since the extract according to the present invention shows dermal tissue regeneration effect, it is useful as a cosmetic product for improving wrinkles.

While the present invention has been described with reference to the particular illustrative embodiment, it is not to be restricted by the embodiment but only by the appended claims. It is to be appreciated that those skilled in the art can change or modify the embodiment without departing from the scope and spirit of the present invention.

1. An extract of Stewartia koreana showing the angiogenesis promoting effect.

2. The extract of Stewartia koreana according to claim 1, which is extracted with any one solvent selected from the group consisting of water, a C₁₋₄ low alcohol, a polar solvent, a non-polar solvent and a mixture thereof.

3. The extract of Stewartia koreana according to claim 1, which is prepared by the following steps:
(a) adding leaves of Stewartia koreana in any one solvent selected from the group consisting of water, a C₁₋₄ low alcohol, a polar solvent, a non-polar solvent and a mixture thereof and performing extraction under reflux at 40 to 80° C. while stirring;
(b) isolating a filtrate by filtration of the extract; and
(c) obtaining a powder by concentrating the filtrate at reduced pressure.

4. The extract of Stewartia koreana according to claim 2, wherein the mixture of solvents is a 30 to 99% ethanol aqueous solution or a 30 to 99% methanol aqueous solution.

5. A pharmaceutical composition for promoting angiogenesis comprising the extract of Stewartia koreana according to claim 1, as an effective ingredient.

6. A pharmaceutical composition for promoting tissue regeneration of a wounded region comprising the extract of Stewartia koreana according to claim 1, as an effective ingredient.

7. A cosmetic for improving wrinkles comprising the extract of Stewartia koreana according to claim 1, as an effective ingredient.

8. The cosmetic according to claim 7, which is formulated into any one selected from the group consisting of skin softener, cream, lotion, skin lotion, pack, foundation, liquid soap, solid soap and cleansing foam.

9. The extract of Stewartia koreana according to claim 2, wherein the extract is purified.


SEQUENCE LISTING (2 sequences)

SEQ ID NO: 1 - DNA, 20 bp, Artificial Sequence
  Synthetic Construct
tgcgcacaaatcccttctac

SEQ ID NO: 2 - DNA, 20 bp, Artificial Sequence
  Synthetic Construct
tgtccctgaacagcccagta